(19) United States
(12) Patent Application Publication
(10) Pub. No.: US 2024/0361254 A1
(43) Pub. Date: Oct. 31, 2024

(54) MOISTURE SENSOR, METHOD OF MEASURING MOISTURE AND METHOD OF MANUFACTURING A COMPONENT FOR MEASURING MOISTURE

(71) Applicant: Freistaat Thüringen (Materialforschungs- und -prüfanstalt an der Bauhaus-Universität Weimar), Weimar (DE)

(72) Inventors: Stefan HELBIG, Weimar (DE); Ralf WAGNER, Erfurt (DE); Frank BONITZ, Weimar (DE); Michael KUHNE, Weimar (DE); Bernd MÜLLER, Erfurt (DE); Alexander ULANOV, Weimar (DE)

(21) Appl. No.: 18/646,692

(22) Filed: Apr. 25, 2024

(30) Foreign Application Priority Data
Apr. 26, 2023 (DE) 10 2023 110 648.1

(51) Int. Cl. G01N 22/04; G01N 33/38

(52) U.S. Cl. CPC G01N 22/04; G01N 33/383

(57) ABSTRACT

The invention relates to a moisture sensor (10) comprising an electromagnetically shielding sensor housing (1) and a microstrip (4) arranged therein as well as at least one coaxial cable (3, 5) each having an inner conductor (3A, 5A) electrically connected to the microstrip (4) and an outer conductor (3B, 5B) electrically connected to the sensor housing (1), wherein the microstrip (4) and the at least one coaxial cable (3, 5) are arranged to conduct microwaves with a frequency (f) in a frequency band (Δf) which covers at least part of the microwave range of between 10 megahertz (MHz) and 300 gigahertz (GHz), the sensor housing (1) is configured as a cavity resonator with a resonant frequency (f_{R1}, f_{R2}, f_{R3}) within the frequency band (Δf) and shielding for the frequency band (Δf) and has openings (2) which establish a hygric contact between the interior and the environment (U) of the sensor housing (1) that is sufficient for moisture equalization, and wherein moisture-storing material is provided in the interior of the sensor housing (1). Furthermore, the invention relates to a method for measuring moisture by means of such a moisture sensor (10) and a method for manufacturing a component comprising such a moisture sensor (10).

### CROSS-REFERENCE TO RELATED APPLICATION

This application claims priority to German application no. 10 2023 110 648.1 filed on Apr. 26, 2023, which is hereby incorporated by reference in its entirety.

### BACKGROUND

### 1. Field of the Invention

According to a first aspect, the invention relates to a moisture sensor set up for measuring the moisture of an environment, comprising an electromagnetically shielding sensor housing and a microstrip arranged therein and further comprising at least one coaxial cable each having an inner conductor electrically connected to the microstrip and an outer conductor electrically connected to the sensor housing. Furthermore, according to a second aspect, the invention relates to a method for measuring moisture using such a moisture sensor. In addition, according to a third aspect, the invention relates to a method for manufacturing a component with a moisture sensor incorporated therein.

### 2. Background Art

Methods known as equilibrium moisture measurement are known from the state of the art for the detection of a moisture load in building components, in which a relative moisture is determined as equilibrium moisture to an adjacent moist solid. In particular, methods known as hygrometric methods for measuring moisture loads in the hygroscopic range are known, for example from Kupfer, K.: Materialfeuchtemessung: Grundlagen, Messverfahren, Applikationen, Normen. Renningen-Malmsheim, expert-Verlag, 1997.

Document DE 10 2005 013 647 B3 relates to a method for measuring the material moisture of a material to be measured by means of a microwave resonator, wherein the material to be measured is introduced into the effective range of the resonator and the material moisture is determined from the change in the quality and the resonator frequency of the resonator, the frequency fed into the resonator being varied and at least the resonance curve of the resonator being swept, the resonator quality factor and the resonant frequency being measured by means of two electrical conductors, one of which is located inside and the other outside the effective range of the resonator, and the frequency-dependent phase difference being measured to determine the resonator quality and the frequency-dependent damping ratio of the two conductors being measured to determine the resonant frequency.

The document DE 10 2017 111 962 A1 describes a method for determining the absolute component moisture. The method comprises the following steps: insertion or application of a sensor device with a moisture sensor in or on a component; non-destructive determination of the air moisture, also known as air humidity with the sensor device at a position in or on the component; and determination of the absolute component moisture from the determined air moisture. Furthermore, a system for the non-destructive determination of the absolute component moisture and the use of an air moisture sensor for the non-destructive determination of the absolute component moisture at a position in or on a component are described.

The document DE 33 39 602 A1 describes a moisture sensor with a microwave transmission line whose dielectric is at least partially formed by the material to be measured with regard to its moisture content and where the attenuation of the microwave energy on the transmission line is measured as a measure of the moisture content. For moisture measurement in the case of anisotropic materials, the transmission line runs along a non-rectilinear path with approximately equal length sections running in a number of different directions, all of which are arranged at equal angular distances above an arc quadrant. The cable path can be designed as a meandering sequence of arc quadrants and the cable can be designed as a slotted cable.

The document DE 33 17 200 A1 describes a method and a device for measuring the moisture of bulk materials, such as moist fine-grained coal, ores, sands or the like, with the characteristic that a line with microwave generator and antenna is made free of wave reflections by adapting the characteristic impedance of the line to the characteristic impedance of the environment of certain moisture, that the antenna is inserted into the moist bulk material to be measured and that when the moisture of the antenna environment changes, part of the energy radiated by the antenna is reflected into the line and measured by a detector.

### SUMMARY

According to its first aspect, the invention is based on the problem of providing an improved moisture sensor for measuring the moisture of an environment.

According to its second aspect, the invention is based on the problem of providing an improved method for measuring moisture.

According to its third aspect, the invention is based on the problem of providing an improved method for manufacturing a component.

According to the invention these problems are solved by the appended claims.

Advantageous embodiments of the invention are the subject of the subclaims.

According to the first aspect of the invention, a moisture sensor comprises an electromagnetically shielding sensor housing with a microstrip arranged therein and with at least one coaxial cable designed to conduct microwaves with a frequency within a predetermined frequency band. The frequency band covers at least a part of the microwave range of between 10 megahertz (MHz) and 300 gigahertz (GHz).

The at least one coaxial cable has an inner conductor and an outer conductor, whereby the inner conductor is electrically connected to the microstrip and the outer conductor is electrically connected to the sensor housing.

The sensor housing is formed as a cavity resonator with a resonant frequency that lies within the predetermined frequency band and is also designed to provide electromagnetic shielding for frequencies in this frequency band. For this purpose, the sensor housing can be made of electrically conductive material and/or coated with an electrically conductive material.

The sensor housing has openings that are provided and set up for a hygric contact, i.e. for a permeability of water and/or water vapor that is sufficient for the moisture compensation between the interior of the sensor housing and its environment. A moisture-storing material is provided in the interior of the sensor housing. The moisture-storing material preferably has a similar moisture storage function (i.e. a similar sorption isotherm) as the material to be measured in the environment of the moisture sensor (being the measurement object). In an embodiment described in more detail below, the moisture-storing material in the interior of the sensor housing is the same as the material in the environment of the moisture sensor.

Moisture (water or water vapor) is transported from the surroundings of the sensor housing into its interior via the hygric contact provided by the openings (e.g. by capillary suction or diffusion) until a moisture balance is established between the interior and the surroundings (the environment) of the sensor housing.

As the sensor housing is electromagnetically shielding (as being manufactured from or coated with electrically conductive materials), electromagnetic waves in the microwave range can be coupled into the sensor housing via the at least one coaxial cable. Said electromagnetic waves propagate inside the sensor housing, but, for practical purposes, do not propagate in its surroundings.

As the sensor housing is designed as a cavity resonator, its resonance frequency is influenced by the dielectric properties inside the sensor housing, which in turn depend on the moisture inside the housing and thus, via the moisture compensation, also on the moisture in the environment.

The moisture sensor according to the invention thus makes it possible to determine the moisture in the environment of the sensor housing by determining the resonant frequency and can be manufactured particularly simply, with low material costs and at low cost. In particular, such a sensor can also be designed to remain permanently in a building component, for example in a building component made from concrete or screed. In addition, a measuring range of particularly high sensitivity for the moisture of the material surrounding the moisture sensor can be set very easily by appropriately selecting the moisture-storing material provided inside the sensor housing, in particular taking into account its moisture storage function.

In one embodiment, a first coaxial cable is led into the sensor housing. Its inner conductor is electrically connected to a first end of the microstrip. A second coaxial cable is also led into the sensor housing and is electrically connected with its inner conductor to a second end of the microstrip that is arranged opposite the first end. The second coaxial cable can be led into the sensor housing through a surface that is opposite to the surface through which the first coaxial cable is led into the sensor housing.

This embodiment enables both transmissive and reflective measurements of the transmission (i.e. forwarding) or reflection of microwaves, respectively. It is therefore particularly versatile and enables a particularly simple and accurate determination of moisture.

In one embodiment, the sensor housing is designed as a rectangular waveguide and has two congruent base surfaces that are arranged parallel to each other (and opposing each other) at a distance that is smaller than the smallest dimension of said base surfaces. The base surfaces are connected to each other via side surfaces of the rectangular waveguide. Openings for moisture equalization between the interior of the sensor housing and its surrounding environment are provided in at least one of the base surfaces. The at least one coaxial cable is guided through one of the side surfaces into the sensor housing, whereby the inner conductor is connected to the microstrip and the outer conductor is connected to the sensor housing.

This design has the advantage of a particularly simple construction.

In a preferred embodiment of the moisture sensor with a sensor housing designed as a rectangular waveguide, a first coaxial cable is designed for coupling in microwaves and is led through a first side surface into the sensor housing and is electrically connected with its inner conductor to a first end of the microstrip. A second coaxial cable is designed for decoupling or forwarding coupled-in microwaves and is led into the sensor housing through a second side surface opposite the first side surface and is electrically connected with its inner conductor to a second end of the microstrip opposite the first end. Alternatively, the second coaxial cable can also be led into the sensor housing through another side surface.

Regardless of the number of coaxial cables led into the sensor housing, a perforation of either one base surface or of both base surfaces (opposite to each other) of the sensor housing is possible for moisture equalization with the environment.

In addition to designing the sensor housing as a rectangular waveguide, embodiments are also possible in which the sensor housing is designed as a waveguide with a different geometry. For example, the sensor housing can be designed as a hollow cylinder in the form of a tube section whose outer surface is perforated. Inside such a hollow cylinder, the microstrip can be formed as a rod made of conductive material or as a printed circuit board with at least one conductor strip arranged on it. The microstrip is preferably arranged centrally along the cylinder axis in the center of the hollow cylinder.

Embodiments of such sensor housing shapes (deviating from a rectangular waveguide) can also be provided with a coaxial cable or with two coaxial cables in order to enable a reflective or a reflective and/or transmissive measurement.

In one embodiment, the microstrip is designed as a copper plate. This embodiment is particularly easy to manufacture.

Alternatively, the microstrip is designed as a copper-coated printed circuit board, preferably with a conductor routing in the form of a single strip line, a multi-strip line or a conductor structure formed therefrom. Such a conductor structure can, for example, be designed as a meandering conductor extending on a printed circuit board (single-strip line) or as a number of meandering conductors arranged parallel to each other (multi-strip line) with a first and a second end.

Alternatively, conductor structures can also be designed as interdigital structures.

Sensor housings provided with this type of microstrip have particularly good resonance behavior, i.e. a resonance frequency that is particularly easy and precise to determine.

In one embodiment, the moisture-storing material inserted into the interior of the sensor housing is selected depending on the material into which the moisture sensor is to be embedded and the moisture of which is to be measured with the moisture sensor. It should have a moisture storage function that is at least similar to the moisture storage function of the object being measured.

A moisture storage function similar to the moisture storage function of the measurement object is understood here and in the following as to mean a moisture storage function according to which the material inside the sensor housing and the material in its environment can each absorb the same amount of volume-related moisture at the same relative moisture (or pore air moisture).

In particular, this similarity or equality relates to the intended measuring range for the sensor (i.e. the range of the associated pore air moisture). The moisture storage functions (of the moisture-storing material inside the sensor housing and of its environment) are also considered similar if the functional curves of the moisture storage functions of the material in the sensor housing and the material in the environment are proportional to each other.

In particular, the material provided in the interior is selected in such a way that its moisture storage function shows an increase of the values of the material moisture along those pore air moisture values, for which the material moisture of the measurement object changes as well. A particularly sensitive and accurate moisture sensor can be provided by selecting the moisture-storing material in such a way that, in a preferred measuring range, small changes in the moisture of the surrounding material cause comparatively large changes in the moisture of the moisture-storing material inside the sensor housing.

Preferably, the moisture-storing material inside the sensor housing is selected as the same material into which the moisture sensor is to be inserted (or embedded) and whose moisture is to be measured (measurement object). The term “same material” (in terms of moisture storage) is used here and in the following also for materials (e.g. for building materials such as screed, mortar or concrete), that are made according to the same formulation as the material in the measurement object (e.g. for building materials given as a mixture of materials produced using the same products and in the same mixing ratio of binder, admixtures/additives or compound, aggregates and added water), but in which the grain size of the aggregates used may differ. The particle size has no significant influence on the moisture storage function.

In such an embodiment, the moisture of the moisture-storing material inside the sensor housing is the same or almost the same as the moisture of the material in its environment, assuming a sufficient moisture equalization between the sensor and its surroundings. This prerequisite has been proven experimentally and by numerical simulation, particularly when measuring the dynamics of moisture transport processes for a drying screed.

This embodiment makes it particularly easy to determine the ambient moisture content by measuring the resonant frequency of the sensor housing.

According to the second aspect of the invention, in a method for measuring moisture by means of a moisture sensor according to the first aspect of the invention, a calibration curve is recorded in a calibration step, which assigns a value of the moisture of the moisture-storing material brought into the sensor housing to at least one waveguide parameter.

As will be explained in more detail below with reference to embodiments, such a waveguide parameter may be selected, for example, as a resonant frequency, a phase difference (between incoming and outgoing microwave signal), an amplitude difference (between incoming and outgoing microwave signal) or as a propagation time (a delay between incoming and outgoing microwave signal), wherein an outgoing microwave signal may be a microwave signal reflected at the moisture sensor or a microwave signal passed through the moisture sensor.

In at least one subsequent measuring step, the value(s) of the at least one waveguide parameter of the sensor housing is/are determined, which is/are influenced by the moisture in its interior, which is set by the moisture equalization with the environment of the sensor housing.

By applying the correspondence (determined in the calibration curve) between the moisture-dependent value of the waveguide parameter and the value of the moisture of the material introduced into the sensor housing, a value of the moisture of the material surrounding the sensor housing is determined.

An advantage of this method is that even without knowledge of an analytical (i.e.: mathematically precisely formulated) relationship between the waveguide parameter(s) of the sensor housing with the microstrip arranged therein and the ambient moisture, an accurate determination of the latter is possible. Further advantages of the method correspond to the advantages of the moisture sensor according to the first aspect of the invention.

In one embodiment of the method, at least one waveguide parameter is selected as the resonance frequency. In the calibration step, a calibration curve is recorded which assigns a value of the moisture content of the moisture-storing material inside the sensor housing to the resonant frequency. In the measurement step, the value of the resonant frequency of the sensor housing is determined, which is influenced by the moisture in its interior, which is driven by the moisture equalization with the environment of the sensor housing. This embodiment has the advantage that a resonance frequency can be easily measured and recorded and evaluated in a calibration curve.

In one embodiment, the resonant frequency is determined by means of a network analyzer as the frequency at which a microwave signal reflected by the moisture sensor has minimum amplitude relative to the amplitude of a microwave signal fed into the moisture sensor (reflective measurement). Alternatively or additionally, when using a moisture sensor provided with a first and a second coaxial cable, the resonant frequency can be determined by means of a network analyzer as the frequency at which a microwave signal transmitted by the moisture sensor has maximum amplitude relative to the amplitude of a microwave signal fed into the moisture sensor (transmissive measurement).

Network analyzers are comparatively inexpensive and therefore enable cost-effective measurement. In addition, network analyzers are available in a mobile design. This means that a single network analyzer can be used to record and evaluate a large number of measuring points, each equipped with a fixed moisture sensor, particularly if a moisture measurement is not to be carried out continuously but only at certain points in time. This enables a particularly cost-saving measurement.

As an alternative to measuring the resonant frequency with a network analyzer, in a method for measuring moisture with a moisture sensor according to the first aspect of the invention, the phase difference and/or the amplitude ratio between a microwave signal reflected by the moisture sensor and/or (if the latter is provided with a first and with a second coaxial cable) a transmitted microwave signal on the one hand and a microwave signal fed into the moisture sensor on the other hand is determined by means of a high-frequency circuit.

In other words: the phase difference and/or the amplitude ratio between the microwave signal fed in via a first coaxial cable on the one hand and the microwave signal reflected on the same coaxial cable (in the case of reflective measurement) or the microwave signal emerging on the second coaxial cable (in the case of transmissive measurement) is/are determined by means of the high-frequency circuit. It is also possible to combine a reflective measurement with a transmissive measurement of the phase difference and/or the amplitude ratio.

High-frequency circuits for determining the phase difference and the amplitude ratio of microwave signals are known from the state of the art.

This method is based on the finding that both variables (i.e. the phase difference and the amplitude ratio) are correlated with the moisture content of the moisture-storing material in the sensor.

In other words, the moisture-dependent microwave line in the moisture sensor is characterized by a phase difference and/or by an amplitude change that the microwave signal experiences during transmission and/or reflection along the microstrip in the moisture sensor. An advantage of this method is that a phase difference and/or an amplitude change can be determined in the time domain and thus independently of a specific resonance frequency. This makes this method particularly flexible and easy to implement.

Furthermore, in a method for measuring moisture using a moisture sensor according to the first aspect of the invention, the propagation time of a microwave signal reflected and/or transmitted by the moisture sensor can also be evaluated by means of a high-frequency frequency circuit.

In other words, the moisture-dependent microwave conduction in the moisture sensor is characterized by a delay (propagation time) which the microwave signal experiences during transmission and/or reflection along the microstrip in the moisture sensor. An advantage of this method is that this delay can be determined for different frequencies which can be selected independently of a resonant frequency or for other signal forms than harmonic signals (for example for pulsed or step signal forms). This makes this method particularly flexible and easy to implement.

High-frequency circuits for determining the propagation time of microwave signals are known from the state of the art.

According to a third aspect of the invention, in a method for manufacturing a component, a moisture sensor according to the first aspect of the invention is introduced into the component or into a mold for manufacturing such component and is surrounded by a material, preferably encapsulated by a flowable material (i.e. embedded into a flowable material). The moisture in the component is then determined according to a method according to the second aspect of the invention.

In this way, the progress of a moisture-reducing process during production, for example a drying or setting process, can be tracked for a precast concrete component, in-situ concrete or screed, and the time at which the component can be used for construction or can be completed in further processing steps can be determined particularly easily and reliably. Alternatively or additionally, the moisture status of a component in use that is exposed to moisture can be continuously determined. The sensor can also be used to detect progressive masonry drying after a renovation measure. Further advantages correspond to the advantages of the moisture sensor according to the first aspect of the invention and the moisture measurement method according to the second aspect of the invention.

In one embodiment of the method for manufacturing a component, the component is manufactured from a flowable material, whereby the same material is provided in the sensor housing as a moisture-storing material, as the material into which the moisture sensor is introduced and embedded in the component. The material can be provided in the sensor housing in a flowable or hardened (non-flowable) state. For example, the material can be cast as a body (cuboid) from the flowable material into which the moisture sensor is inserted before it is placed in the sensor housing. The hardened body (cuboid) is inserted as a solid body into the interior of the sensor housing.

Alternatively or additionally, the sensor housing is at least partially constructed using this solid body. Usage of the same material inside the sensor housing as well as the hygric contact mediated by the openings in the sensor housing ensure that the moisture content of the moisture-storing material in the sensor housing is the same as the moisture content of the flowable material from which the component is made.

This means that the moisture inside the component can be determined particularly easily and accurately.

In one embodiment, the flowable material is a screed material, a mortar (e.g. in the case of masonry renovation), a tile adhesive, concrete or another flowable material for the production of structural components or joints. Such structural components can, for example, be designed as prefabricated concrete parts, but also as components that are made of in-situ concrete or similarly integrated directly into a structure.

The components provided with a moisture sensor according to this embodiment can be monitored particularly well during their completion, for example during the hardening of a tile adhesive or during the hydration of a component made of concrete.

For example, the time at which a load-bearing capacity is reached or the time at which subsequent work steps can be started can be determined particularly accurately and reliably. In addition, components manufactured according to this embodiment of the method are suitable for use in installation environments exposed to moisture, as they can be continuously monitored for moisture ingress by means of the integrated moisture sensor.

### BRIEF DESCRIPTION OF THE DRAWINGS

Examples of embodiments of the invention are explained in more detail below with reference to drawings, wherein:

FIG. 1 schematically shows a resonant sensor housing with coaxial cables connected at both ends,

FIG. 2 schematically shows a resonant sensor housing with a coaxial cable connected at one end, and

FIG. 3 schematically shows the frequency-dependent reflected amplitude response for microwaves coupled into a resonant sensor housing.

Corresponding parts are marked with the same reference signs in all figures.

### DETAILED DESCRIPTION OF SPECIFIC EMBODIMENTS

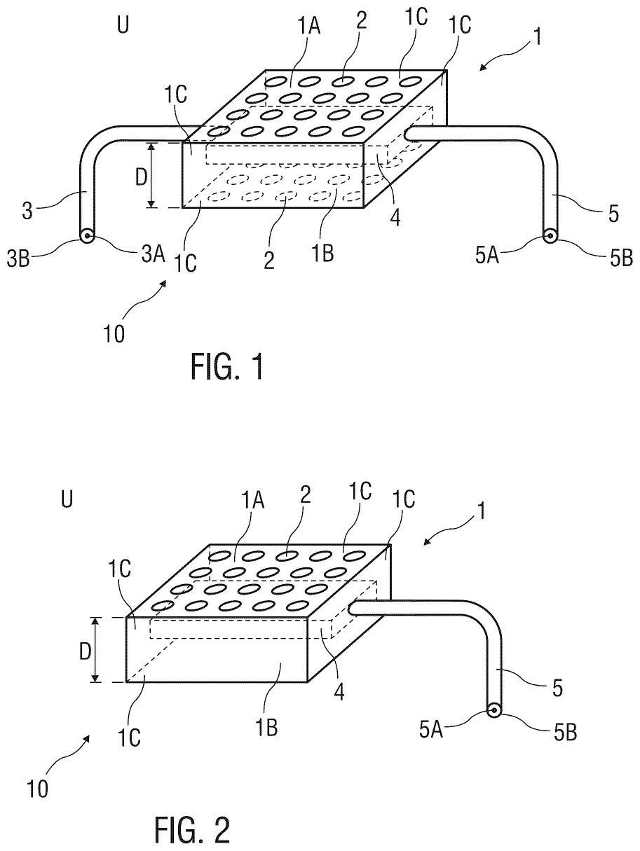

FIG. 1 schematically shows a moisture sensor 10 with a cuboid sensor housing 1 with a housing structure that is resonant for electromagnetic waves. The sensor housing 1 is formed by metallic, electrically conductive material or by outer surfaces 1A, 1B, 1C coated with such a material. This results in almost complete shielding of the electromagnetic waves that are coupled into the interior of the sensor housing 1.

The cuboid sensor housing 1 has an upper base surface 1A and an opposing lower base surface 1B arranged parallel to it, which are electrically connected to each other via side surfaces 1C. The base surfaces 1A, 1B are spaced at a distance D which is small in relation to each of the two longitudinal extensions of the base surfaces 1A, 1B. For example, the distance D is less than one tenth of the smallest longitudinal extension of the base surfaces 1A, 1B.

Openings 2 are formed in the base surfaces 1A, 1B and optionally also in the side surfaces 1C. Said openings 2 are permeable to moisture such that water or water vapor can pass between the interior of the sensor housing 1 and its environment U. In the present case, the openings 2 are circular, but other geometric shapes are also possible.

The extension (in this case the diameter) of each opening 2 is small compared to the extension of the sensor housing 1 and also small compared to the wavelength of the electromagnetic waves for which coupling into the sensor housing 1 is provided, so that electromagnetic waves do not leak from the interior of the sensor housing 1 into the environment U, or only to a very small extent.

In particular, such a sensor housing 1 can, in good approximation, be regarded as a rectangular waveguide if the extension (i.e. the footprint) of each opening 2 is small compared to the wavelength of electromagnetic waves coupled into the sensor housing 1. This ensures that the sensor housing 1 practically completely shields electromagnetic waves with a sufficiently long wavelength (relative to the size of the openings 2) from the environment U.

For example, if the electromagnetic waves intended for coupling are microwaves with a frequency f of between 1500 Megahertz and 3000 Megahertz, the openings 2 can be designed as circular openings with a diameter of between one and two millimeters.

To transmit the electromagnetic waves into/out of the sensor housing 1, a first coaxial cable 3 is connected to a microstrip 4 arranged inside the sensor housing 1. The microstrip 4 is designed in the present case as a copper plate or copper-coated plate and is arranged coplanar and approximately centrally between the base surfaces 1A, 1B. Alternatively, the microstrip 4 can also be designed as a two-, three- or multi-strip line, for example as a correspondingly etched copper-coated printed circuit board.

The microstrip 4 is electrically connected to the inner conductor 3A of the first coaxial cable 3. The outer conductor 3B of the first coaxial cable 3 is electrically connected to the outer surfaces 1A, 1B, 1C of the sensor housing 1. The inner conductor 3A is preferably led through one of the side surfaces 1C of the sensor housing 1.

Similarly, the microstrip 4 and the outer surfaces 1A, 1B, 1C are connected to a second coaxial cable 5. The inner conductor 5A of the second coaxial cable 5 is preferably led through the side surface 1C opposite the side surface 1C at which the inner conductor 3A of the first coaxial cable 3 passes. The outer conductor 5B of the second coaxial cable 5 is electrically connected to the outer surfaces 1A, 1B, 1C of the sensor housing 1.

All four complex-valued scattering parameters (input reflection factor S11, backward transmission factor S12, forward transmission factor S21 and output reflection factor S22) can be measured on a sensor housing 1 designed as a cavity resonator in this way. While S11 and S22 describe the reflected signals for the first coaxial cable 3 and the second coaxial cable 5 respectively, S12 represents the transmitted signal from the first coaxial cable 3 to the second coaxial cable 5 and S21 represents the transmitted signal from the second coaxial cable 5 to the first coaxial cable 3.

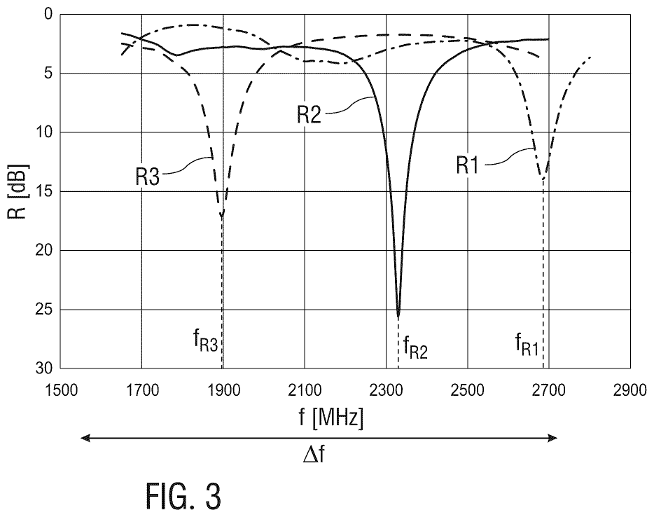

A cavity resonator is formed by the opposing metallic side surfaces 1C with the coaxial cables 3, 5, which are essentially electrically conductive over a large area. The resonant frequency f_{R1}, f_{R2}, f_{R3} of said cavity resonator is determined by the geometry of the sensor housing 1 and by the material inside the sensor housing 1, in particular by its electrical permittivity, as will be explained in more detail below with reference to FIG. 3.

FIG. 2 shows a further embodiment of a resonant sensor housing 1 in which, in contrast to the embodiment shown in FIG. 1, only one (in this case the upper) base surface 1A is perforated, i.e. provided with openings 2, while the opposite (in this case the lower) base surface 1B is formed as a solid metallic surface or as a metallically coated surface. Furthermore, the embodiment shown in FIG. 2 has only a second coaxial cable 5, while the first coaxial cable 3 connected opposite to the second coaxial cable 5 in the embodiment according to FIG. 1 is omitted here. Via said remaining second coaxial cable 5, electromagnetic waves are coupled in and the reflected electromagnetic waves are coupled out, i.e. measured.

This embodiment is easier to manufacture and install, as only a single (in this case: the second) coaxial cable 5 needs to be routed and connected to the sensor housing 1. For this embodiment with only one coaxial cable 5, only the scattering parameter S11 (input reflection factor) can be determined, with which the task at hand can be solved in principle.

The space above the microstrip 4 inside the sensor housing 1 can either be initially empty or be initially filled with material that is suitable for absorbing and/or storing moisture. If the interior is initially provided as an empty space, then when the sensor housing 1 is embedded in an environment U with sufficiently flowable material, for example screed material, mortar, tile adhesive, concrete, material for filling or leveling floors or similar material, this material can penetrate into the interior of the sensor housing 1 via the openings 2, which is an intended embodiment. Alternatively, the sensor housing 1 can also be filled with this material from the environment U initially, i.e. before embedding.

In a particularly advantageous embodiment, all material below the microstrip 4 up to the lower base surface 1B is formed from printed circuit board material which is chosen as to have a moisture absorption capacity that is as low as possible. In other words, a printed circuit board provided with a microstrip 4 on its upper side, which is not drawn in detail in FIG. 2, is arranged with its opposite underside directly abutting the inner side of the lower base surface 1B, for example glued and/or soldered with said inner side.

The free space between the top of the printed circuit board and the inside of the upper base surface opposite it can be empty or filled with moisture-storing material.

Moisture entering this space influences the waveguide in the moisture sensor 10. In the event that the free space above the circuit board is initially empty, it can be designed to hold a similar or preferably the same material (e.g. screed, mortar, concrete) as is present in the environment of the sensor.

Preferably, a microstrip 4 is applied to a printed circuit board with a conductive coating on both sides. The upper (top) side of the printed circuit board, which is etched at least in the edge area, then forms the microstrip 4 (which is electrically connected to the inner conductor of the at least one coaxial cable 3, 5). Alternatively, a (for example meander-shaped) conductor structure can also be etched on the top side. The underside of the printed circuit board becomes part of the shielding sensor housing by being mechanically and electrically connected to it (e.g. soldered, optionally also glued).

The metallic (or metallically coated) upper part of the sensor housing 1 (i.e. the space above the top of the printed circuit board that encloses the moisture-storing material) is electrically conductively connected to the electrical ground of the at least one coaxial cable 3, 5 (i.e. to its respective outer conductor 3B, 5B) and to the underside of the printed circuit board (for example by soldering) in such a way that no open slots remain. This forms a mechanically particularly strong microstrip 4, which can be mounted and adjusted very easily.

The invention is based on the finding that the interaction of electromagnetic waves, particularly in the microwave range, preferably in a frequency range between 1 Gigahertz and 6 Gigahertz, is essentially determined by the moisture inside the sensor housing 1. The good electromagnetic shielding of the sensor housing 1 ensures that the interaction of these electromagnetic waves with the environment U of the sensor housing 1 is low and can be neglected (i.e. no or negligible leakage of electromagnetic waves).

Essentially, the interaction of electromagnetic microwave radiation with moisture inside the sensor housing 1 is determined by the continuous alignment of the dipoles in the alternating electromagnetic field, which removes energy from the incoming microwave radiation and converts it into heat. The energy of the electromagnetic waves that are reflected in the sensor housing 1 and returned via the first coaxial cable 3 and/or transmitted via the second coaxial cable 5 is reduced accordingly.

FIGS. 1 and 2 are merely examples of the basic design of a moisture sensor 10. Deviating from this, it is also possible, for example, to combine the sensor housing 1 shown perforated at the top and bottom in FIG. 1 with only one of the two coaxial cables 3, 5 shown in FIG. 1 as shown in FIG. 2. Other geometries of a cavity resonator formed by the sensor housing 1 are also conceivable, for example a cylindrical geometry.

By way of example only and purely schematically, FIG. 3 shows the frequency-dependent logarithmic attenuation curve of electromagnetic waves which are coupled into the sensor housing 1 via the first coaxial cable 3, radiated by the microstrip 4 into the interior of the sensor housing 1 and which interact with water molecules in said interior. If the sensor housing 1 only has a one (e.g. a second) coaxial cable 5 according to the embodiment shown in FIG. 2, a major part of the energy coupled into the sensor housing 1 is not transmitted to the water molecules, and is thus reflected via the second coaxial cable 5.

The proportion of the reflected wave energy depends on the frequency f of the microwaves and is particularly low for a resonance frequency f_{R1}, f_{R2}, f_{R3}. In other words: microwaves coupled in at or nearby the resonant frequency f_{R1}, f_{R2}, f_{R3} interact to a particularly high degree with the water molecules inside the housing and are reflected and/or (if a first coaxial cable 3 would be connected) transmitted to a correspondingly low degree.

It turned out that the resonance frequency f_{R1}, f_{R2}, f_{R3} depends on the moisture inside the sensor housing 1 and shifts towards lower frequencies with increasing moisture. Due to the almost complete shielding of the electromagnetic waves by the sensor housing 1, the material and its moisture in the environment U of the sensor housing 1 has no or only a negligibly small direct effect on the resonant frequency. f_{R1}, f_{R2}, f_{R3}. The resonance frequency is also f_{R1}, f_{R2}, f_{R3} not influenced by structures (for example metallic objects or different layers of material) in the environment U. However, the moisture of the environment U does of course influence the moisture inside the sensor housing 1 through diffusion exchange and thus indirectly determines the value of the resonant frequency f_{R1}, f_{R2}, f_{R3}.

FIG. 3 illustrates a first to third frequency-dependent reflection curve R1 to R3 of a reflection coefficient R, specified as a logarithmic reflection coefficient in decibels (dB). Each reflection curve R1 to R3 is assigned a resonance frequency f_{R1}, f_{R2}, f_{R3} as that frequency f at which the reflection curve R1 to R3 reaches its respective minimum. The first reflection curve R1 with the first resonance frequency f_{R1} is associated with low moisture (inside the sensor housing 1). The second reflection curve R2 with the second resonance frequency f_{R2} is associated with medium moisture and the third reflection curve R3 with the third resonance frequency f_{R3} is assigned to high moisture (with otherwise unchanged material inside the housing).

The respective value of the resonant frequency f_{R1}, f_{R2}, f_{R3} can be determined by measuring the reflected signal (via the first coaxial cable 3) and/or by measuring the transmitted signal (via the second coaxial cable 5, if present), for example using a network analyzer. Inexpensive network analyzers, with which a frequency-dependent reflection curve R1 to R3 over a frequency band Δf can be measured, are known from the state of the art, for example the product LibreVNA as an open source vector network analyzer. Here, the frequency band Δf must be selected so that it covers the assigned resonance frequency f_{R1}, f_{R2}, f_{R3} for each of the at least one expected moisture values.

The position of the resonant frequency f_{R1}, f_{R2}, f_{R3} can alternatively or additionally also be determined by measurement methods in which not only the amplitude of the reflected and/or transmitted microwave signal, but also its phase position (in relation to the phase position of the microwave signal coupled in via the first coaxial cable 3) is evaluated. For example, the phase shift (i.e. the offset of the phase positions of the fed-in microwave signal on the one hand and the reflected or forwarded microwave signal on the other hand) that is observed at a certain, predetermined frequency f (for example at a frequency of 300 Megahertz), the position of the resonant frequency f_{R1}, f_{R2}, f_{R3} can be determined.

With this approach, it is not necessary to determine the reflection coefficient R across the whole frequency band Δf. Thereby, while a high-frequency circuit is required to determine the phase difference between the coupled and reflected microwave signal, a complete network analyzer can be saved. This means that the moisture of the environment U can be determined with a particularly simple and cost-saving measuring device.

To assign a moisture value to a resonance frequency f_{R1}, f_{R2}, f_{R3} (determined using a network analyzer, for example, or alternatively using a high-frequency circuit), a calibration will prove helpful. In a calibration, the moisture value of a certain material inside the sensor housing 1 is varied and the resulting position of the resonant frequency f_{R1}, f_{R2}, f_{R3} dependent on the variable moisture value is determined. is determined. The result of the calibration can be captured as a calibration curve, with which each subsequently determined resonance frequency f_{R1}, f_{R2}, f_{R3} is assigned a moisture value for the relevant material inside the housing.

If the material inside the housing of the sensor housing 1 is the same material as in the environment U to be measured, the moisture value of the environment U can be determined directly using this calibration curve if good hygric contact is established between the inside of the sensor housing 1 and the environment U through the openings 2 in the sensor housing 1, for example by placing the sensor housing 1 into a component made of a flowable material during its production. Due to the good hygric contact, the moisture between the interior of the sensor housing 1 and the component in its environment U is balanced out (i.e. equalized).

Since the moisture storage properties inside the housing and in the surrounding area U are the same (due to the same material), i.e. since, given the same pore air moisture or the same capillary pressure, the same material moisture is produced, the moisture value inside the housing determined using the measured resonant frequency f_{R1}, f_{R2}, f_{R3} by application of the calibration curve corresponds to the moisture value in the material of the component surrounding the sensor housing 1.

If a material with known moisture storage properties is provided in the interior of the sensor housing 1, which differs from the material in the environment U (for example in a construction component in which the sensor housing 1 was brought into), the moisture potential (i.e. the capillary pressure or pore air moisture) can be determined by measuring the resonance frequency. f_{R1}, f_{R2}, f_{R3}. According to methods for measuring equilibrium moisture known from the state of the art, the material moisture can also be determined from this capillary pressure or from this pore air moisture by using the moisture storage function of the material in the environment U of the sensor housing 1.

The electromagnetically shielded sensor housing 1 has the effect, that coupled electromagnetic fields, in particular microwaves, are not emitted from the sensor housing 1 or are only emitted to a negligible extent. The value of the resonant frequency f_{R1}, f_{R2}, f_{R3} is thus determined by the moisture content of the moisture-storing material inside the sensor housing 1. The resonant frequency f_{R1}, f_{R2}, f_{R3} will not change in conjunction with the hygric coupling of the measurement object in the environment U. Also, the dielectric properties of the material in the environment U and the geometric dimensions of such a material do not influence the resonant frequency determined using, for example, the reflected or transmitted microwave signal f_{R1}, f_{R2}, f_{R3}. Interaction between the interior of the sensor housing 1 and its environment U only occurs through moisture exchange via the openings 2.

In a further application, the sensor housing 1 can also be used to determine the moisture, particularly in environments U with very high moisture, for example with a relative moisture of over 95 percent. In this case, it will prove helpful to introduce a material into the interior of the sensor housing 1 that has a high moisture storage capacity for very high moisture values.

### LIST OF REFERENCES

1 sensor housing
1A upper base surface, outer surface
1B lower base surface, outer surface
1C side surface, outer surface
2 opening
3, 5 first, second coaxial cable
3A, 5A inner conductor
3B, 5B outer conductor
4 microstrip
10 moisture sensor
D distance
f frequency
f_{R1}, f_{R2}, f_{R3} first to third resonant frequency
Δf frequency band
R reflectance
R1, R2, R3 first to third reflection curve
U environment

What is claimed is:

1. A moisture sensor comprising an electromagnetically shielding sensor housing, a microstrip arranged in said sensor housing and at least one coaxial cable each having an inner conductor electrically connected to the microstrip and an outer conductor electrically connected to the sensor housing,
the microstrip and the at least one coaxial cable configured to conduct microwaves with a frequency in a frequency band which covers at least a part of the microwave range of between 10 megahertz (MHz) and 300 GHz,
wherein the sensor housing is configured as a cavity resonator with a resonant frequency within the frequency band and as shielding for said frequency band and wherein the sensor housing has openings which establish a hygric contact between its interior and an environment of the sensor housing that is sufficient for moisture equalization, and wherein moisture-storing material is provided in the interior of the sensor housing.

2. The moisture sensor according to claim 1, wherein a first coaxial cable is led into the sensor housing and is electrically connected by its inner conductor to a first end of the strip line, and a second coaxial cable is led into the sensor housing and is electrically connected by its inner conductor to a second end of the strip line opposite the first end.

3. The moisture sensor according to claim 1, wherein the sensor housing is designed as a rectangular waveguide and has two congruent base surfaces which are arranged opposite one another in parallel at a distance which is smaller than the smallest dimension of the base surfaces, wherein the base surfaces are connected via side surfaces, wherein openings for moisture equalization are made in at least one base surface and wherein at least one coaxial cable is led into the sensor housing through one of the respective side surfaces.

4. The moisture sensor according to claim 1, wherein the microstrip is designed as a copper plate.

5. The moisture sensor according to claim 1, wherein the microstrip is designed as a copper-coated printed circuit board with a line routing formed as a single-strip line or as a multi-strip line or as a conductor structure comprising such single-strip line and/or such multi-strip line.

6. The moisture sensor according to claim 1, wherein the material provided in the interior of the sensor housing has the same or a similar moisture storage function as the material in the environment to be measured.

7. A method for moisture measurement with a moisture sensor according to claim 1, wherein
in a calibration step a calibration curve is captured which assigns a moisture value of the material provided in the sensor housing to at least one waveguide parameter and
in at least one subsequent measuring step the value of said at least one waveguide parameter is measured and a moisture value of the environment is determined by applying the calibration curve to said measured waveguide parameter.

8. The method according to claim 7, wherein at least one waveguide parameter is recorded as a resonant frequency in the calibration step and at least on value of said resonant frequency is determined in the at least one subsequent measurement step.

9. The method according to claim 8, wherein the resonant frequency is determined by means of a network analyzer as the frequency at which a microwave signal reflected and/or transmitted by the moisture sensor has an extreme amplitude relative to the amplitude of a microwave signal fed into the moisture sensor.

10. The method according to claim 7, wherein at least one waveguide parameter is determined by means of a high-frequency circuit as the phase difference and/or as the amplitude ratio of a microwave signal reflected and/or transmitted by the moisture sensor, on the one hand, and of a microwave signal fed into the moisture sensor, on the other hand.

11. The method according to claim 7, wherein the transit time of a microwave signal transmitted and/or reflected by means of the moisture sensor is evaluated by means of a high-frequency circuit.

12. A method of manufacturing a component, wherein a moisture sensor is introduced into the component, the moisture sensor comprising an electromagnetically shielding sensor housing, a microstrip arranged in said sensor housing and at least one coaxial cable each having an inner conductor electrically connected to the microstrip and an outer conductor electrically connected to the sensor housing, the microstrip and the at least one coaxial cable configured to conduct microwaves with a frequency in a frequency band which covers at least a part of the microwave range of between 10 megahertz (MHz) and 300 GHz,
wherein the sensor housing is configured as a cavity resonator with a resonant frequency within the frequency band and as shielding for said frequency band and wherein the sensor housing has openings which establish a hygric contact between its interior and an environment of the sensor housing that is sufficient for moisture equalization, and wherein moisture-storing material is provided in the interior of the sensor housing,
the method comprising:
filling a flowable material into the component such that the moisture sensor is embedded in the flowable material, and
thereafter determining the moisture in the component by a method according to claim 7,
wherein the material introduced into the sensor housing of the moisture sensor is the same as the material in which the moisture sensor is embedded in the component.

13. The method according to claim 12, wherein the material provided in the sensor housing is flowable and is designed as screed material, mortar, tile adhesive, concrete or as a flowable material for the production of building components.
